(19) United States
(12) Patent Application Publication
(10) Pub. No.: US 2004/0089303 A1
(43) Pub. Date: May 13, 2004

(54) Nose filter device

(76) Inventor: Dennis Chien, Chungho City (TW)

Correspondence Address: Alan D. Kamrath, Rider Bennett, LLP, Suite 2000, 333 South Seventh Street, Minneapolis, MN, 55402, US

(21) Appl. No.: 10/693,747

(22) Filed: Oct. 23, 2003

(30) Foreign Application Priority Data
Nov. 11, 2002 (TW) 091132995

(51) Int. Cl. A61M 16/00; A62B 7/10

(52) U.S. Cl. 128/206.11; 128/205.27

(57) ABSTRACT

A nose filter device has a holder and multiple filters, the holder has multiple holes defined in the holder and the filters are mounted in the holes. When a user wears the nose filter device, the air can pass from the holes and through the filters, and then moves into the nose of the user. Because the air passes through the filters, the air can be cleaned by the nose filter device.

### BACKGROUND OF THE INVENTION

1. Field of the Invention

The present invention relates to a nose filter device, more particularly to a disposable nose filter device that can be easily worn and which totally covers the face area around the nostril to filter the air.

2. Description of Related Art

With increasing global affluence, many people have given up walking or riding bicycles in favor of motorized transportation. In particular, some nations have developed a culture where motorcycles and motor scooters are the vehicle of choice. The drawbacks are that the 2 and 4 stroke vehicles produce enormous quantities of toxic gases, foul odor, sooty deposits etc and the riders are directly exposed to the contaminants while car passengers etc are at least shielded somewhat from the worst effects. To try to reduce some of the harmful effects of the pollution, many riders wear simple masks that cover the mouth and nose. However, those masks have the following drawbacks:

1. The mask is not convenient for a motorcycle rider who wears a motorcycle helmet because when the motorcycle helmet is taken off, the mask often becomes unintentionally removed as well.

2. There are many gaps between the periphery of the mask and the users face whereby non-filtered air can be inhaled by the wearer.

3. The mask covers the user's mouth and nose whereby the mask is exposed to damp exhaled air which creates an ideal breeding ground for bacteria, and so contradicts the health and safety target of the mask.

4. The cost of the mask is not cheap, so the mask is often used more than one time. Some users will wash the mask to keep the mask clean, but the filtrating efficiency of the washed mask is diminished. The present invention provides a nose filter to mitigate or obviate the aforementioned problems.

### SUMMARY OF THE INVENTION

The primary objective of the present invention is to provide a nose filter device that has a holder and multiple filters, the holder has multiple holes defined in the holder and the filters are mounted on the holes. When a user wears the nose filter device, the air can pass from the holes and through the filters, and then moves into the nose of the user. Because the air passes through the filters, the air can be cleaned by the nose filter device.

Other objects, advantages and novel features of the invention will become more apparent from the following detailed description when taken in conjunction with the accompanying drawings.

### BRIEF DESCRIPTION OF THE DRAWINGS

FIG. 1A is a perspective view of a first embodiment of a nose filter device in accordance with the present invention;

FIG. 1B is a side plan view of the nose filter device in FIG. 1A;

FIG. 1C is a bottom plan view of the nose filter device in FIG. 1A;

FIG. 1D is a perspective view of a second embodiment of the nose filter device in accordance with the present invention;

FIG. 1E is a perspective view of a third embodiment of the nose filter device;

FIG. 1F is a perspective view of a fourth embodiment of the nose filter device;

FIG. 2 is a perspective view of a fifth embodiment of the nose filter device;

FIG. 3A is a perspective view of a sixth embodiment of the nose filter device with a filter inside a tube;

FIG. 3B is a plan view of a sixth embodiment of the nose filter device in FIG. 3A;

FIG. 4 is a plan view of a sixth embodiment of the nose filter device with three filters inside a tube;

FIG. 5 is a plan view of a sixth embodiment of the nose filter device with three filters separated inside a tube;

FIG. 6 is a plan view of a sixth embodiment of the nose filter device with multiple protrusions formed inside; and

FIG. 7 is a plan view of a seventh embodiment of the nose filter device.

### DETAILED DESCRIPTION OF THE PREFERRED EMBODIMENT

The nose filter device in accordance with the present invention has a holder and multiple filters. It is to be appreciated that more than one type of holder can be fitted to fix filters. The holders have at least two holes defined through the holders and can be pastes, plates, net plates, cover (such as cloth or paper) and tubes in the present invention. The filters are mounted inside the holder and face the holes on the holder. The holder with the filter can be attached to a user to clean the air to be breathed.

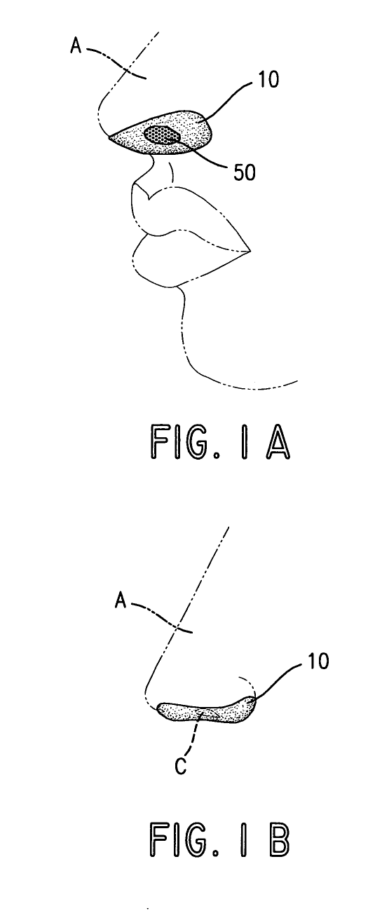

With reference to the series of FIGS. 1A to 1C, a first embodiment of a nose filter device in accordance with the present invention is shown and has the holder and multiple filters (50). The holder in the first embodiment is a paste (10). The paste (10) can be pasted around the nose (A) and has two side surfaces (not numbered) and multiple holes (not numbered). One of the side surfaces can adhere to a user. The filter (50) is fixed on peripheries defining the holes of the paste (10) and the filter (50) is fixed on the side surface that can adhere to the user's nose (A). With reference to FIG. 1C, the holes of the paste (10) with the filter (50) are under the nostrils (C) of the user so that the air can pass through the holes and the filter (50). According to the way to use the present invention, the air firstly passes through the nose filter device then the particles in the air will attach to the filter and the filtered air passes into the nose. Referring to FIG. 1D, a second embodiment of the nose filter device is shown and has the holder and a large filter (50′). The holder in the second embodiment is a large piece of paste (10′). The structure of the second embodiment is almost the same as the first embodiment, but the nose filter device of the second embodiment can cover the nose (A) completely. The large paste (10′) with the large filter (50′) can adhere to a user from the ala to the philtrum so that the nose filter device can separate the dirty air completely.

With reference to FIG. 1E, a third embodiment of the nose filter device has the holder and a filter (50). The holder in the third embodiment is two plates (11). Each plate (11) has two holes (not numbered) defined through the plate (11), and the filter (50) is mounted between the plates (11).

With reference to FIG. 1F, a fourth embodiment of the nose filter device is shown and the plates (11) can be replaced with net plates (12). Because the air can pass through the net plates (12), the net plates (12) do not define through holes such as the holes on the plates (11). The net plates (12) can be used for separating large particles in the air and one side of the net plates (12) can attach to a user.

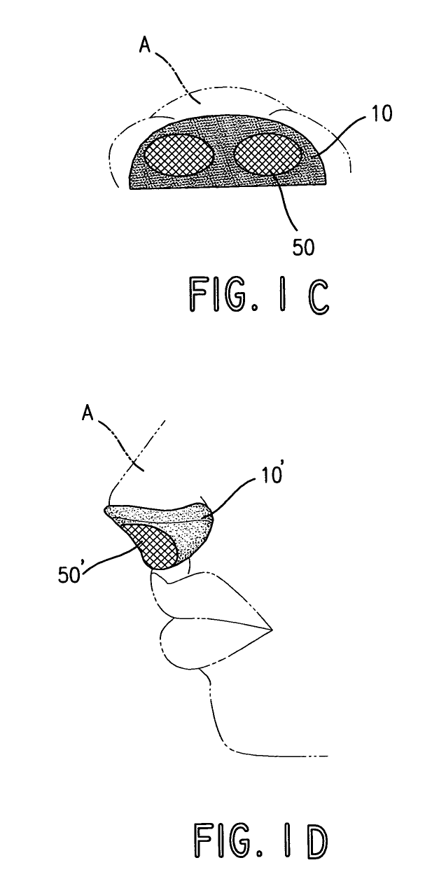

With reference to FIG. 2, a fifth embodiment of the nose filter device has the holder and multiple filters (50). The holder in the fifth embodiment is a cover (20). The cover (20) can be made of cloth or paper and shaped as cup-like with an opening (not numbered). The opening is held under the nostrils and can be made with elastic force to provide a snugly-fitting hermetic seal. The nose filter device of fifth embodiment also can be used as FIG. 1D to cover a user's nose from the ala to the philtrum.

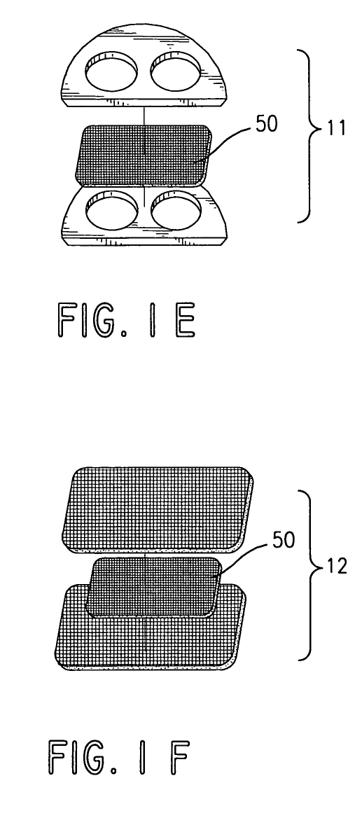

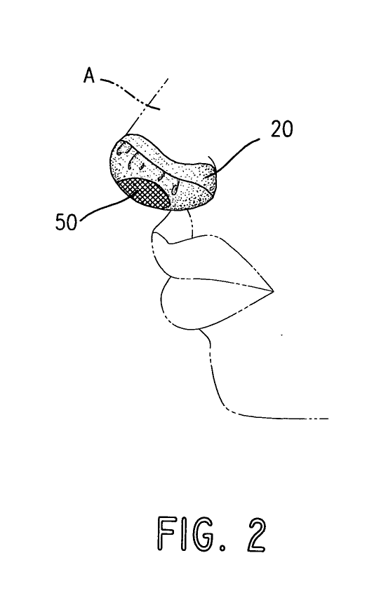

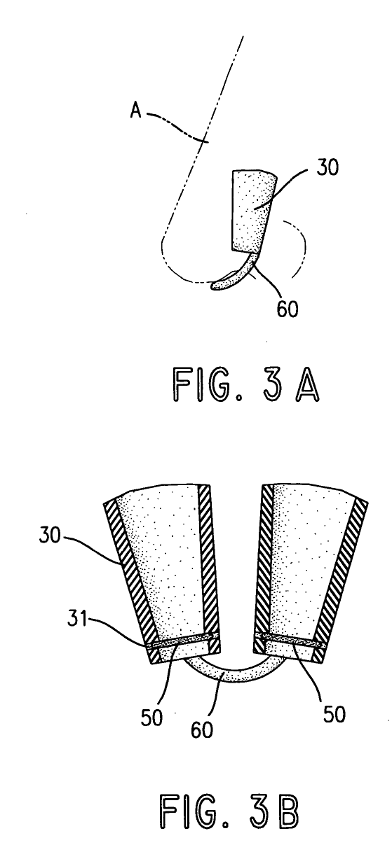

With reference to FIGS. 3A and 3B, and FIG. 4 to FIG. 6, a sixth embodiment of the nose filter device has the holder and multiple filters (50). The holder in sixth embodiment is two tubes (30,30′,34). The tubes (30,30′,34) are made of elastic material and can be received inside the nostrils. Each tube (30,30′,34) has a central passage (not numbered) for the air to pass through. Each tube (30,30′,34) has an inner end (not numbered), an outer end (not numbered), an inner surface (not numbered) and a recess (31). The recess (31) is defined in the inner surface near the outer end of the tube (30,30′,34). One or more of the above filters (50) is mounted inside one of the recesses (31). Each recess (31) further comprises multiple separators to form multiple chambers (32). Each chamber (32) contains one filter (50). The nose filter device in the sixth embodiment further comprises a connector (60) to connect two tubes (30,30′,34). When a user wears the nose filter device of the sixth embodiment, the nose filter device of the sixth embodiment can be simply taken off by pulling the connector (60) back and the connector (60) can prevent the tubes (30,30′,34) from moving inside the nose (A).

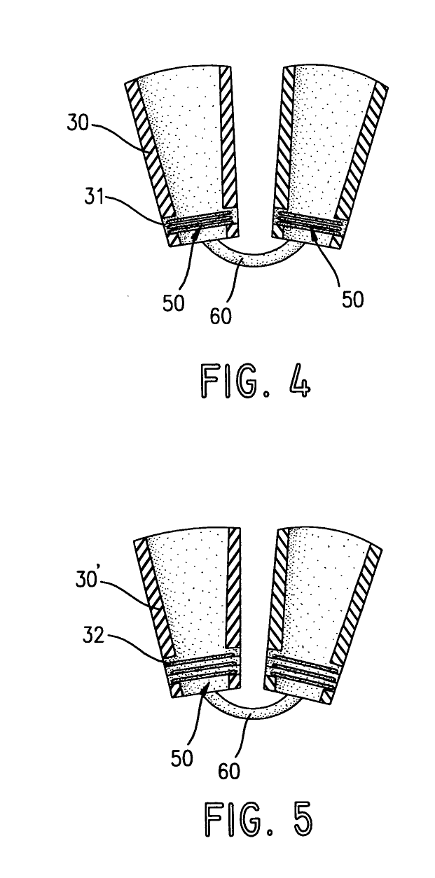

With reference to FIG. 6, each tube (34) of the nose filter device further comprises multiple protrusions (33) formed on the inner surface of the tube (34) so that the central passage of each tube (34) is like a human nose.

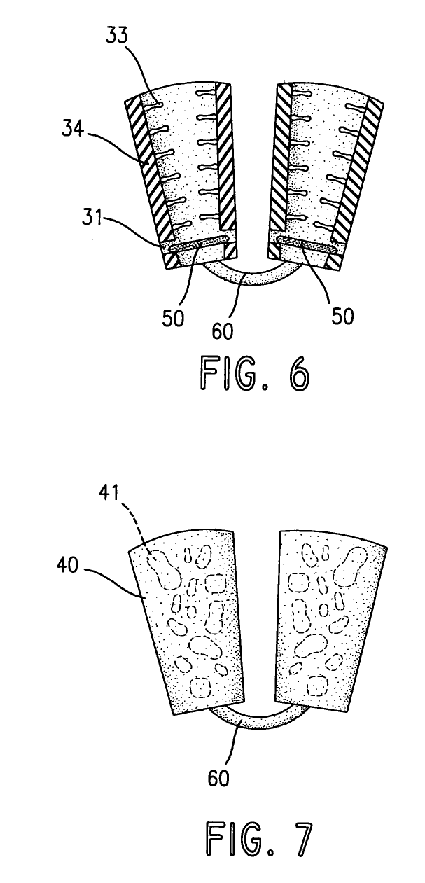

With reference to FIG. 7, a seventh embodiment of the nose filter device has two tubes (40) and the connector (60) mounted between the tubes (40). The tubes (40) are also made of the elastic material and each tube (40) has multiple chambers (41) such as sponge formed inside. Each chamber (41) is communicated with each other. When a user wears the nose filter device in the seventh embodiment, the air will pass through the chambers (41) then pass into the user's nose and the passing air is clean.

In the first to the sixth embodiments, each tube further comprises a safety net to prevent the filters from moving inside the nose.

Prior to the full blown symptoms of a cold, a person's nose becomes dry. When a user wears the nose filter device in the present invention, the user can add some medication and water to the filters (50) and the tube (40) to minimize the development of the cold. Furthermore, when a person is tired, aroma-therapeutic essence can be added to the nose filter device to promote the user's sense of well-being. Some persons that have nasal catarrh may exhale an odor but by wearing the nose filter device with fragrance added thereto, the odors can be disguised whereby the user can feel more confident and people need not experience the unpleasant odors. In the foregoing embodiments, the pastes and the tubes can be made of a material that can save heat to contain heating inside the nose filter device.

To add interest to the nose filter device, a sounder can be mounted on each tube. When a child wears the nose filter device with the sounders, the exhaust air can make the sounds from the sounders so that the child's parents locate the child.

Even though numerous characteristics and advantages of the present invention have been set forth in the foregoing description, together with details of the structure and function of the invention, that the disclosure is illustrative only, and changes may be made in detail, especially in matters of shape, size, and arrangement of parts within the principles of the invention to the full extent indicated by the broad general meaning of the terms in which the appended claims are expressed is to be understood.

What is claimed is:

1. A nose filter device having
a holder adapted to a nostril of a nose and having
at least two holes defined through the holder; and
multiple filters mounted on the holder and facing the holes in the holder.

2. The nose filter device as claimed in claim 1, wherein the holder is a paste and has two side surfaces, one of the side surfaces is adapted to adhere to a user.

3. The nose filter device as claimed in claim 1, wherein the holder comprises two plates, the filter is mounted between the two plates.

4. The nose filter device as claimed in claim 3, wherein the plates are net plates.

5. The nose filter device as claimed in claim 1, wherein the holder is a cover with an opening.

6. The nose filter device as claimed in claim 5, wherein the cover is made of paper.

7. The nose filter device as claimed in claim 5, wherein the cover is made of cloth.

8. The nose filter device as claimed in claim 1, wherein the holder comprises two tubes, each tube has one hole, an inner end, an outer end and an inner surface, and the filters are mounted respectively inside the tubes near the outer end.

9. The nose filter device as claimed in claim 8, wherein a connector is mounted between the outer ends of the tubes.

10. The nose filter device as claimed in claim 8, wherein each tube has a recess defined in the inner surface of the tube near the outer end, and the filters are mounted respectively inside the recesses of the tubes.

11. The nose filter device as claimed in claim 9, wherein each tube has a recess defined in the inner surface of the tube near the outer end, and the filters are mounted respectively inside the recesses of the tubes.

12. The nose filter device as claimed in claim 10, wherein three filters are mounted inside the recess of each tube.

13. The nose filter device as claimed in claim 12, wherein each recess has multiple separators formed inside the recess to form multiple chambers, each filter is received inside a respective one of the chambers.

14. The nose filter device as claimed in claim 8, wherein each tube has multiple protrusions formed on the inner surface of the tube.

15. The nose filter device as claimed in claim 8, wherein each tube has multiple chambers defined inside the tube, each chamber is communicated with each other.
